(19) United States
(12) Patent Application Publication
(10) Pub. No.: US 2026/0174872 A1
(43) Pub. Date: Jun. 25, 2026

(54) CYCLODEXTRIN ACID BASE SOLUBILIZATION OF POORLY WATER-SOLUBLE SMALL MOLECULES

(71) Applicant: ST. JOHN'S UNIVERSITY, Queens, NY (US)

(72) Inventors: Mimansa Goyal, Queens, NY (US); Vivek Gupta, Queens, NY (US)

(21) Appl. No.: 18/999,939

(22) Filed: Dec. 23, 2024

(51) Int. Cl. A61K 47/40; A61K 31/445; A61K 31/496; A61K 47/12; A61K 47/22

(52) U.S. Cl. CPC A61K 47/40; A61K 31/445; A61K 31/496; A61K 47/12; A61K 47/22

(57) ABSTRACT

A composition contains a BCS Class II or IV drug that is acid-base solubilized with a pharmaceutically acceptable acid, and which is complexed with β-cyclodextrin. The β-cyclodextrin complexed drug exhibits enhanced bioavailability. The drug may be formulated for oral administration and/or to exhibit rapid release when administered. Preferable compositions may contain no crosslinked or polymerized non-active components.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION BY REFERENCE STATEMENT

Not Applicable

### STATEMENT REGARDING PRIOR DISCLOSURE BY THE INVENTOR OR A JOINT INVENTOR

Not Applicable

### BACKGROUND OF THE INVENTION

### 1. Field of the Invention

The present invention relates to cyclodextrin-based acid-base solubilization of poorly water-soluble molecules. More particularly, the present invention relates to cyclodextrin-based acid-base solubilization of drugs belonging to either BCS-Class II or BCS-Class IV.

### 2. Brief Description of the Background Art

Cyclodextrins have been used with acids to create drug delivery systems. Prior examples of drug delivery systems include those taught, for instance, in Ghorpade et al., Citric acid crosslinked yclodextrin/hydroxypropylmethylcellulose hydrogel films for hydrophobic drug delivery, Int. J. Biolog. Macromol., Vol. 93, Part A, (2016) 75-86, which relates to hydrogel-based drug delivery and drug-controlled release within the hydrogel matrix. It uses crosslinked hydrogels with 3-CD and citric acid but does not combine the solubilization mechanism through direct acid-base reactions to enhance bioavailability.

Escobar et al., Coatings of Cyclodextrin/Citric-Acid Biopolymer as Drug Delivery Systems: A Review, Pharmaceutics, Vol. 15, No. 1 (2023) 296 uses cyclodextrin/citric-acid coatings for localized delivery from implant surfaces, and targets localized release rather than systemic bioavailability.

Sarafska et al., Enhanced Solubility of Ibuprofen by Complexation with β-Cyclodextrin and Citric Acid, Molecules, Vol. 28, No. 7 (2024) 1650 relates to enhancing delivery of the weak acid ibuprofen by forming a complex using mechanical milling.

Anand et al., Citric acid-γ-cyclodextrin crosslinked oligomers as carriers for doxorubicin delivery, Photochem. Photobiol. Sci., Vol. 12 (2013) 1841-54 uses citric acid to conduct chemical reactions creating a crosslinked 3D network of polymerized cyclodextrins and obtain a matrix that can retain doxorubicin.

Karpkird et al., A novel chitosan-citric acid crosslinked beta-cyclodextrin nanocarriers for insoluble drug delivery, Carbo. Res., Vol. 498 (2020) 108184 forms nanoparticles using crosslinking reactions between citric acid and β-cyclodextrin, followed by the incorporation of chitosan using an ionic gelation process. Nanoparticles are formed for controlled release of curcumin.

### SUMMARY OF THE INVENTION

It is an object of the invention to provide a drug delivery vehicle that permits increased delivery of poorly-soluble drugs.

It is another object of the invention to provide formulations with improved bioavailability and which are not sustained-release or delayed-release.

It is an additional object of the invention to provide improved bioavailability drug formulations for oral delivery and which are rapid-release.

It is yet another object of the invention to provide drug formulations having improved bioavailability and which contain no crosslinked or polymerized non-active components.

It is a further object of the invention to provide formulations of drugs belonging to BCS Class II or to BCS Class IV that are prepared in the presence of pharmaceutically acceptable acids and β-cyclodextrin to achieve improved delivery and bioavailability.

These objects and others are achieved by the present invention, which relates to the cyclodextrin-based acid-base super solubilization of poorly-soluble drugs.

### BRIEF DESCRIPTION OF THE DRAWINGS

The accompanying drawings, which are incorporated in and constitute a part of the specification, illustrate embodiments of the invention and, together with the description, serve to explain principles of the invention. The patent or application file contains at least one drawing executed in color. Copies of this patent or patent application publication with color drawing(s) will be provided by the Office upon request and payment of the necessary fee.

FIG. 1 shows acid-base solubilization of nintedanib in citric acid and various different cyclodextrin derivatives.

FIG. 2 illustrates permeability of Nin, Nin-Acid, NCD, and NACD in intestinal cells: Caco2.

FIG. 3 portrays the evaluation of permeation potential of NACD formulation using 3D Epi-Intestinal Tissue modelling.

FIG. 4 illustrates the in-vitro safety assessment of Nin-Acid, NCD and ACD on intestinal epithelial cells and preclinical assessment of NACD via oral route.

FIG. 5 shows acid-base solubilization of haloperidol in ascorbic acid and various different cyclodextrin derivatives.

FIG. 6 illustrates an in-vitro safety assessment of Halo, Halo-Acid, Halo-CD and HACD on intestinal epithelial cells and preclinical assessment of HACD via oral route.

### DETAILED DESCRIPTION OF THE INVENTION

The Biopharmaceutics Classification System (BCS) is a four-class system for differentiating drugs on the basis of their solubility and permeability. This system restricts the prediction using the parameters solubility and intestinal permeability. The solubility classification is based on a United States Pharmacopoeia (USP) aperture.

The four BCS classes highlight the limiting factors of the absorption process: Class I, high-solubility high-permeability drugs, indicate the easier and straightforward development process, and complete absorption is expected; Class II, low-solubility high-permeability drugs, indicate that a solubility/dissolution limitation is expected; Class III, high-solubility low-permeability drugs, indicate that the intestinal absorption of this class of drugs will be limited by their permeability rate; and Class IV, low-solubility low-permeability drugs, having very poor oral bioavailability and which are inclined to exhibit very large inter- and intrasubject variability.

This invention relates to cyclodextrin-based acid-base solubilization of poorly water-soluble drugs, and preferably to the cyclodextrin-based acid-base solubilization of poorly water-soluble drugs that are weakly basic. The cyclodextrin-based acid-base solubilization technique has shown improved bioavailability of these drugs, as it enhances their dissolution and absorption rates. Accordingly, administering cyclodextrin-based acid-base solubilized drugs according to the present invention allow for lower drug dosages to be administered and still receive therapeutic benefit. Lower therapeutic drug administration levels beneficially reduce the risks of drug-induced toxicity as well as the risks of side effects associated with these drugs.

In our invention, we demonstrate the cyclodextrin-based acid basic solubilization of hydrophobic small molecules to improve their bioavailability and therapeutic efficacy. We demonstrate the efficacy of this these formulations using models for idiopathic pulmonary fibrosis. Idiopathic pulmonary fibrosis (IPF) is a serious chronic disease that affects the tissue surrounding the air sacs, or alveoli, in the lungs. This condition develops when that lung tissue becomes thick and stiff for unknown reasons. Over time, these changes can cause permanent scarring in the air sacs or alveoli in the lungs, called fibrosis, making it progressively more difficult to breathe.

In this study, we used nintedanib and haloperidol. Nintedanib (Nin) is a small molecule tyrosine kinase inhibitor that binds to a family of growth factor receptors and prevents the proliferation of fibroblasts. Nin belongs to BCS class IV, portraying low aqueous solubility and low intestinal permeability. Nin is a P-glycoprotein (P-gp) efflux substrate and possesses an oral bioavailability of 4.7%.

Haloperidol (Halo) is a first-generation typical antipsychotic and is commonly used to block dopamine D2 receptors in the brain and exert its antipsychotic action. Haloperidol is also shown to inhibit the activation of myofibroblasts, which are a key part of the fibrotic process. Halo is a BCS class-II molecule with poor aqueous solubility.

The present invention relates to formulations of BCS Class-II and BCS class-IV using cyclodextrins. Cyclodextrin (CD) is a family of cyclic oligosaccharides, e.g., sugar molecules bound together to form rings of different sizes, and are enzymatically produced from starch.

Specifically, the sugar units are called glucopyranosides, which are glucose molecules that exist in the pyranose (six-membered) ring configuration. The three natural types of cyclodextrin are α-, β-, and γ-cyclodextrin, which are respectively made up of 6, 7, and 8 glucose units.

Cyclodextrin is biocompatible and low toxicity. Cyclodextrin has a hydrophobic center and a hydrophilic surface, which allows it to encapsulate hydrophobic compounds without losing its water solubility.

We demonstrate these formulations using 2-hydroxypropyl-β-cyclodextrin. 2-Hydroxypropyl-β-cyclodextrin (HP-β-CD) has the structure

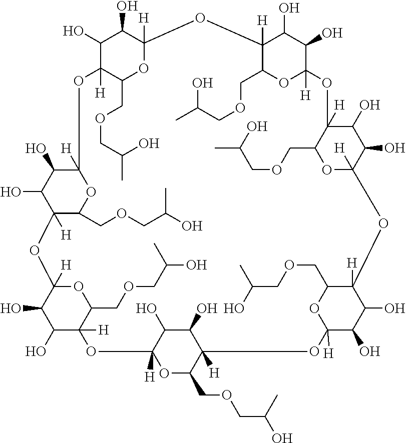 [SMILES: CC(O)COCC1OC2OC3C(COCC(C)O)OC(OC4C(COCC(C)O)OC(OC5C(COCC(C)O)OC(O[C@H]6[C@H](O)C(O)[C@@H](OC7C(COCC(C)O)OC(OC8C(COCC(C)O)OC(OC1C(O)[C@H]2O)[C@H](O)C8O)[C@H](O)C7O)O[C@@H]6COCC(C)O)[C@H](O)C5O)[C@H](O)C4O)[C@H](O)C3O]

We also demonstrate these formulations using sulfobutylether-β-cyclodextrin. Sulfobutylether-β-cyclodextrin (SBE-β-CD) has the structure

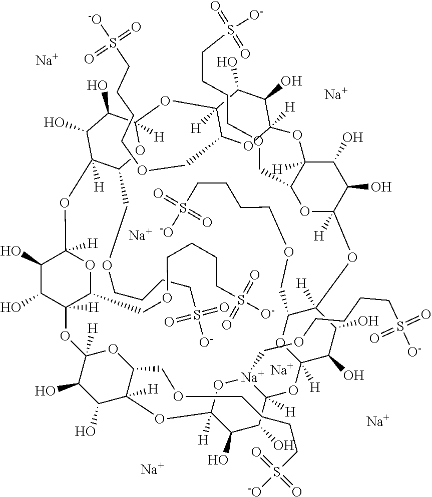

### Example 1: Acid-Base Solubilization of Nintedanib with Cyclodextrin

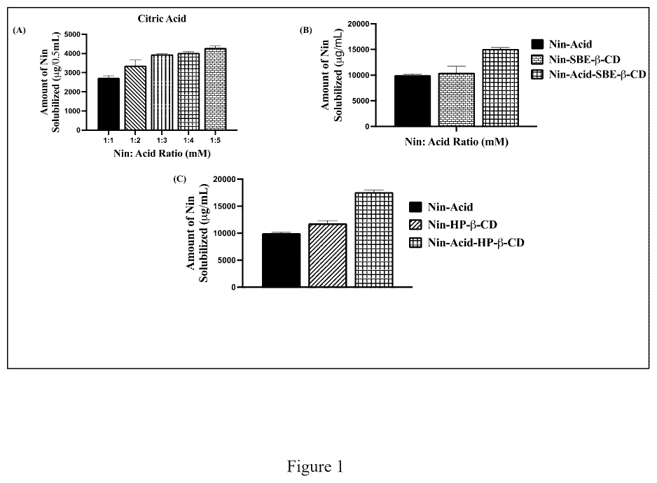

We demonstrate that all acids increased Nin solubility compared to water with citric acid, showing the highest solubility of Nin among the acids evaluated, with a concentration-dependent increase in solubility up to 4.3±0.1 mg/mL at a 1:5 mM concentration. (FIG. 1A.) We also show that incorporating Nin-Acid into a cyclodextrin complex improves its solubility and facilitates permeation through the intestinal membrane using HP-β-CD and SBE-β-CD. Specifically, Nin-Acid shows a significantly increased solubility of 15.1±0.3 mg/mL with SBE-β-CD. (FIG. 1B.) Nin-Acid further shows a significantly increased solubility of and 17.4±0.4 mg/mL with HP-β-CD. (FIG. 1C.) The findings teach that formation of an inclusion complex with these derivatives significantly enhances the solubility of Nin-Acid. Data Represents mean±SD of n=3 individual trials.

### Example 2: Permeability Potential of Nintedanib Through a CaCO-2 Monolayer

We evaluate the permeability potential of plain Nin, Nin-Acid (NA), Nin-Cyclodextrin (NCD) and Nin-Acid-Cyclodextrin (NACD) for three hours on intestinal epithelial cells: using a CaCO-2 monolayer. CaCO-2 monolayer is a widely used cell line for permeability assays. (Kus et al., CaCO-2 Cell Line Standardization with Pharmaceutical Requirements and In Vitro Model Suitability for Permeability Assays, Pharmaceutics, Vol. 15, No. 11 (2023) 2523.)

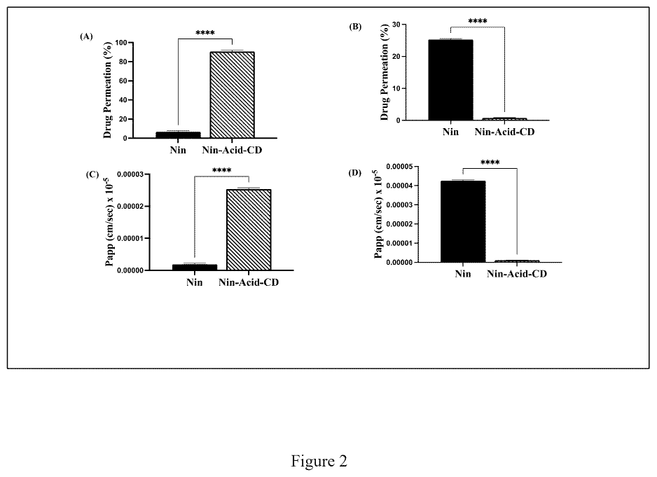

At 40 μg/ml, NACD demonstrated a significantly higher permeation from apical to the basolateral side of 90.5±1.3%, yielding a 13.6-fold increase in permeation compared to plain Nin (6.6±1.2%). (FIG. 2A.) In contrast, the Nin permeation in NACD from the basolateral to the apical side was remarkably lower than plain Nin, accounting for 0.6±0.03% and 25.2±0.2%, respectively. (FIG. 2B.)

The apparent permeability (Papp) from apical to basolateral and basolateral to apical was also calculated and plotted based on the permeation of Nin and NACD across the intestinal membrane. FIG. 2C illustrates the Papp from apical to basolateral. As illustrated therein, the Papp of plain Nin for apical to basolateral was 1.8×10⁻⁶±3.4×10⁻⁷ and the Papp of NACD 2.5×10⁻⁵±3.7×10⁻⁷, Papp for basolateral to apical is illustrated in FIG. 2D. As seen therein, the Papp for plain Nin was 4.2×10⁻⁵±4.4×10⁻⁷, and the Papp for NACD was 1.1×10⁻⁶±5.2×10⁻⁸. The efflux ratio of Nin was 2.2×10⁻¹, whereas the efflux ratio of NACD was 4.6×10⁻². These findings show that forming inclusion complexes using acid-base solubilization and cyclodextrins enhances the oral delivery of Nin. (****p<0.000l) depicts a significant difference between Nin and NACD formulation. Data represents n=3 trials.

### Example 3: Permeability Potential of Nintedanib Using an Epi-Intestinal Tissue Model

We further assessed the permeability potential of plain Nin and NACD through the Epi-Intestinal Tissue Model. Epi-Intestinal tissue is produced from normal human cells and provides a highly differentiated 3D tissue model that closely recapitulates the physiology, tissue structure and function of the small intestine epithelium, and provides a more holistic model for investigation drug absorption and metabolism. (Markus et al., Human small intestinal organotypic culture model for drug permeation, inflammation, and toxicity assays, In Vitro Cell Dev. Biol. Anim., Vol. 57, No. 2 (2021) 160-73.)

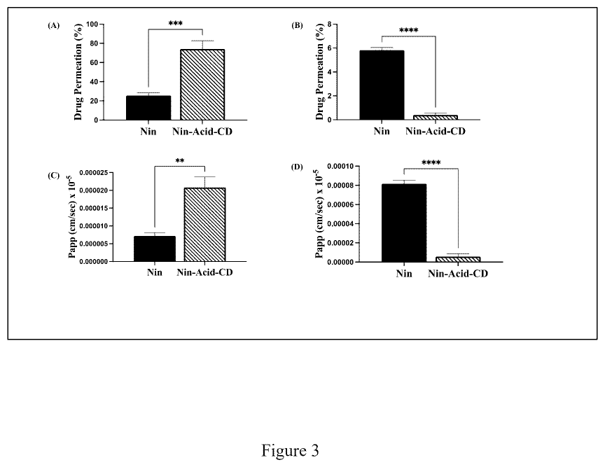

As shown in FIG. 3A, NACD demonstrated significantly higher permeation from apical to basolateral, accounting for 74.1±8.5%, than plain Nin (25.5±2.7%), corresponding to a 2.9-fold increase in Nin's permeation. Conversely, the drug permeation (%) from basolateral to apical direction (FIG. 3B) was 0.4±0.1% (NACD) and 5.8±0.2% (plain Nin), representing a 14.5-fold reduction in drug permeation from NACD. FIG. 3C illustrates that the Papp for apical to basolateral was lower for plain Nin (7.1×10⁻⁶±9.5×10⁻⁷) than NACD (2.0×10⁻⁵±2.9×10⁻⁶), while Papp from basolateral to apical direction was higher in the case of plain Nin (8.1×10-5±3.6×10-6) compared to NACD (5.7×10-6±2.8×10-6). (FIG. 3D.) Finally, the efflux ratio calculations showed that Nin displayed a ratio of 1.1×10⁺¹ whereas NACD portrayed a ratio of 2.7×10⁻¹. **p<0.01, ***p<0.001, where ****p<0.0001 depicts a significant difference between Nin and NACD formulation. Data represents n=3 trials.

These results demonstrate that NACD increases Nin's transport across the intestinal membrane while concurrently reducing P-glycoprotein (P-gp) efflux. We attribute the increased permeability of Nin to the enhanced aqueous solubility and inhibition of P-gp activity by HP-β-CD.

### Example 4: Safety Assessment of Nin in Intestinal Epithelial Cells and Rats

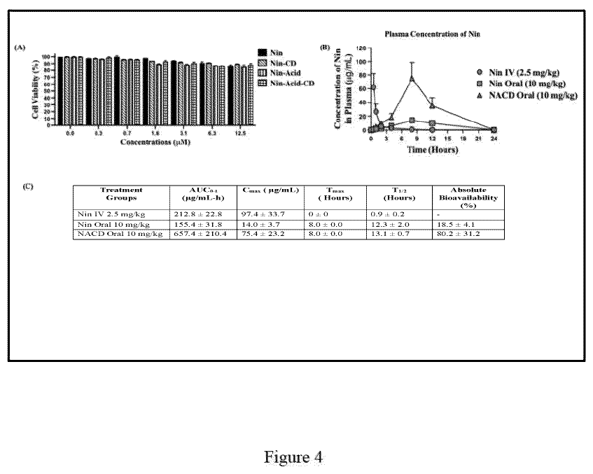

The safety assessment of four formulations (Nin, Nin-Acid, NCD, and NACD) was conducted for three hours on intestinal epithelial cell line CaCO-2. The study revealed that none of the formulations were toxic to the cells even at varying concentrations. (FIG. 4A.) These findings are significant as they demonstrate the safety and biocompatibility of the developed formulations, which is a crucial factor for their use in clinical trials and future commercialization.

Pre-clinical studies were conducted in Male Sprague-Dawley rats to determine the pharmacokinetic parameters of NACD. Plasma concentration of Nin in rats was measured at different time intervals after intravenous and oral administration, and of NACD after oral administration. The results of the experiment are presented in FIG. 4B, where the plasma concentration of Nin is expressed in μg/mL as a function of time in hours for Nin (IV: 2.5 mg/kg), Nin (Oral: 10 mg/kg), and NACD (Oral: 10 mg/kg). The results show that intravenous administration of Nin resulted in a maximum plasma concentration of 62.0±19.8 μg/mL within 0.5 hours, followed by a linear decrease to 2.7±0.4 μg/mL within 4 hours.

The pharmacokinetic parameters of intravenous Nin were calculated, which included an AUC of 212.8±22.81 μg/mL-h, a Cₘₐₓ of 97.4±33.7 μg/mL, and a Tₘₐₓ of 0±0 hours. (FIG. 4C.) The area under the plasma drug concentration-time curve (AUC) is the definite integral of the concentration of a drug in blood plasma as a function of time and reflects the actual body exposure to drug after administration of a dose of the drug.

The half-life (T_{1/2}) of intravenous Nin was calculated to be 0.8±0.2 hours. In contrast, oral administration of plain Nin resulted in a lower plasma concentration, with only 0.7±1.0 μg/mL reaching the systemic circulation at 0.5 hours due to its low aqueous solubility and P-gp-mediated efflux. The Cₘₐₓ of plain Nin was 14.0±3.7 μg/mL at a Tₘₐₓ of 8.0±0.0 hours. (FIG. 4C.)

The AUC and T_{1/2} of plain Nin were 155.4±31.8 μg/mL-h and 12.3±2.0 hours, respectively (FIG. 4C) and the absolute bioavailability of oral Nin was calculated at 18.5±4.1%. However, NACD showed a significantly higher plasma concentration of Nin, with a concentration of 2.7±1.0 μg/mL within 0.5 hours. (FIG. 4B.) The Cₘₐₓ of Nin with NACD was remarkably enhanced, with a value of 75.4±23.2 μg/mL at a Tₘₐₓ of 8.0±0.0 hours, yielding a 3.6-fold increment in the concentration of Nin in the systemic circulation. The AUC was 657.6±210.3 μg/mL-h, accounting for a 4.35-fold enhanced AUC for NACD compared to plain Nin. Although no significant increase in the T_{1/2} was seen, NACD portrayed a T_{1/2} of 13.1±0.7 hours. Finally, the absolute bioavailability of NACD was 80.2±31.2%, corresponding to a ˜4.3-fold higher bioavailability in comparison to plain Nin. Data represents mean±SD of n=4 rats/group.

### Example 6: Acid-Base Solubilization of Haloperidol

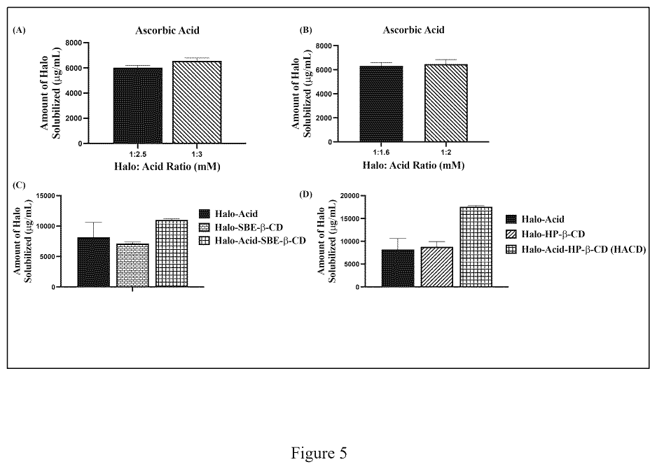

The study examined the solubility of Halo at two different concentrations, 20 and 30 mM, in five different acids having a concentration of 50 and 60 mM, respectively. Ascorbic acid showed the highest solubility of Halo, corresponding to ˜6 mg/mL (50 mM) and 6.4 mg/mL (60 mM), as shown in FIG. 5A and FIG. 5B, respectively. Since no significant difference in the solubility of Halo was observed with 50 mM and 60 mM concentrations of ascorbic acid, 50 mM ascorbic acid concentration was finalized as optimum to enhance the solubility of Halo. We then formulated an inclusion complex of Halo-Acid with SBE-β-CD and HP-β-CD.

FIG. 5C and FIG. 5D show the solubility of excess Halo (50 mM) in the presence of cyclodextrin alone and acid plus cyclodextrin. Halo-Acid demonstrate a significantly increased solubility of 11.0±0.2 mg/mL with SBE-β-CD (FIG. 5C) and 17.5±0.2 mg/mL with HP-β-CD. (FIG. 5D.)

### Example 7: Safety Assessment of Halo in Intestinal Epithelial Cells and Preclinical Bioavailability Assessment in Rats

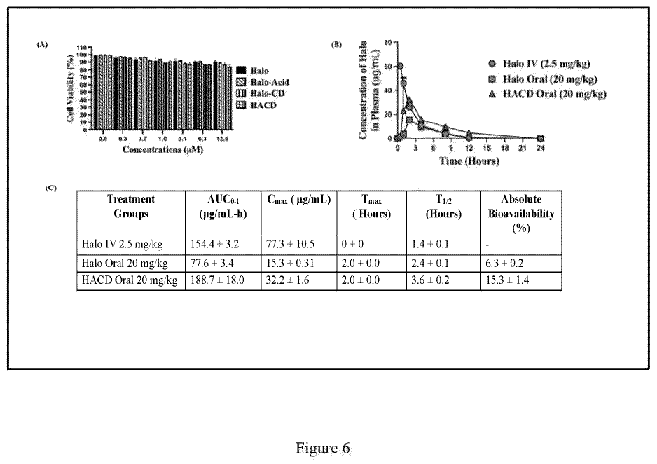

Four formulations (Halo, Halo-Acid, Halo-CD, and HACD) were assessed for safety on intestinal epithelial cell line CaCO-2. The optimized inclusion complex and all the excipients used in the formulations were found to be nontoxic to the intestinal epithelial cells, demonstrating cell viability of >90% after 3 hours of treatment. (FIG. 6A.) We also investigated how HACD affects the pharmacokinetic parameters of Halo in pre-clinical studies by measuring the plasma concentration of Halo in rats at different time intervals after administering it intravenously and orally, or by administering it orally with HACD.

Plasma concentration of Halo expressed in μg/mL as a function of time in hours for Halo (IV: 2.5 mg/kg), Halo (Oral: 20 mg/kg), and HACD (Oral: 20 mg/kg). The results are provided in FIG. 6B and show that intravenous administration of Halo produced a maximum plasma concentration of 60.0±1.5 μg/mL within 0.5 hours, followed by a linear decrease to 10.6±1.1 μg/mL within 4 hours. The pharmacokinetic parameters of intravenous Halo include an AUC of 154.4±3.2 μg/mL-h, an estimated Cₘₐₓ of 77.3±10.5 μg/mL, and a Tₘₐₓ of 0±0 hours. (FIG. 6C.) The half-life (T_{1/2}) of intravenous Halo was calculated to be 1.4±0.1 hours.

On the other hand, plain Halo taken orally resulted in a lower plasma concentration of Halo, with only 1.3±0.1 μg/mL reaching the systemic circulation due to its low aqueous solubility. The Cₘₐₓ of plain Halo was 15.3±0.31 μg/mL at a Tₘₐₓ of 2.0±0.0 hours. Additionally, the AUC and T_{1/2} of plain Halo were 77.6±3.4 μg/mL-h and 2.4±0.1 hours, respectively. (FIG. 6C.) The absolute bioavailability of oral Halo was calculated at 6.3±0.2%.

HACD showed a significantly higher plasma concentration of Halo, with a concentration of 2.4±0.3 μg/mL within 0.5 hours. (FIG. 6B.) The Cₘₐₓ of Halo with HACD was remarkably enhanced, with a value of 32.2±1.8 μg/mL at a Tₘₐₓ of 2.0±0.0 hours, resulting in a 2.1-fold increase in the concentration of Halo in the systemic circulation. The AUC was 188.7±18.0 μg/mL-h, accounting for a 2.4-fold enhanced AUC for HACD compared to plain Halo. A significant increase in the T_{1/2} was seen, and HACD portrayed a T_{1/2} of 3.6±0.2 hours. Finally, the absolute bioavailability of HACD was 15.3±1.4%, resulting in a ˜2.5-fold higher bioavailability than plain Halo. Data represents mean±SD of n=4 rats/group.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

We claim:

1. A composition, comprising:
a BCS Class II or IV drug that is acid-base solubilized with a pharmaceutically acceptable acid, and which is complexed with β-cyclodextrin, wherein
the BCS Class II or IV drug exhibits enhanced bioavailability.

2. The composition according to claim 1 wherein the pharmaceutically acceptable acid is citric acid, ascorbic acid, maleic acid, glutaric acid or tartaric acid.

3. The composition according to claim 2, wherein the β-cyclodextrin is 2-hydroxypropyl-β-CD.

4. The composition according to claim 3, wherein the pharmaceutically acceptable acid is citric acid.

5. The composition according to claim 4, wherein the drug is nintedanib.

6. The composition according to claim 3, wherein the pharmaceutically acceptable acid is ascorbic acid.

7. The composition according to claim 6, wherein the drug is haloperidol.

8. The composition according to claim 2, wherein the β-cyclodextrin is 2-sulfobutylether-β-CD.

9. The composition according to claim 8, wherein the pharmaceutically acceptable acid is citric acid.

10. The composition according to claim 9, wherein the drug is nintedanib.

11. The composition according to claim 8, wherein the pharmaceutically acceptable acid is ascorbic acid.

12. The composition according to claim 11, wherein the drug is haloperidol.

13. The composition according to claim 1, further comprising a comestible excipient, and which is formulated for oral delivery, wherein
said drug is rapidly released upon ingestion.

14. The composition according to claim 1, wherein said BCS Class II or IV drug is weakly basic.

15. The composition according to claim 1, which contain no crosslinked or polymerized non-active components.
